(19) United States
(12) Patent Application Publication
(10) Pub. No.: US 2008/0076742 A1
(43) Pub. Date: Mar. 27, 2008

(54) METHODS AND COMPOSITIONS FOR TREATING DISEASES ASSOCIATED WITH NEOVASCUALRIZATION

(75) Inventors: Nader Sheibani, Madison, WI (US); Daniel M. Albert, Madison, WI (US)

Correspondence Address: BAKER DONELSON BEARMAN CALDWELL & BERKOWITZ, PC, 555 11TH STREET, NW, 6TH FLOOR, WASHINGTON, DC, 20004, US

(73) Assignee: WISCONSIN ALUMNI RESEARCH FOUNDATION, Madison, WI (US)

(21) Appl. No.: 11/869,074

(22) Filed: Oct. 9, 2007

Related U.S. Application Data
(63) Continuation-in-part of application No. 11/552,439, filed on Oct. 24, 2006.
(60) Provisional application No. 60/731,684, filed on Oct. 31, 2005.

(51) Int. Cl. A61K 31/59; A61P 27/02

(52) U.S. Cl. 514/167

(57) ABSTRACT

Methods and compositions for treating pathologies resulting from neovascular growth in the eye such as those manifested as retinopathy of prematurity, diabetic retinopathy and macular degeneration. The invention comprises the administration of an effective amount of vitamin or a salt, prodrug or derivative thereof, administered at doses less than toxicity and results in a significant reduction in angiogenesis or the formation of neo-vascular growth. The invention can be used to treat existing diseases or prophylactically to treat those at risk.

### CROSS-REFERENCE TO RELATED APPLICATIONS

This application is continuation-in-part of U.S. patent application Ser. No. 11,552,439, which claims the benefit of U.S. Provisional Application No. 60/731,684, filed Oct. 31, 2005, both of which are incorporated herein be reference in their entirety.

### STATEMENT OF GOVERNMENT INTEREST

This invention was made in part with support from the National Institutes of Health under Grant No. EY013700 and EY001917. The Government of the United States of America has certain rights in this invention.

### FIELD OF THE INVENTION

This invention is generally directed to methods and compositions for treating eye diseases with neovascularization component, especially those associated with choroidal neovascularization or retinal neovascularization. More particularly the invention recites the use of Vitamin D analogues or derivatives to prevent or inhibit neovascularization.

### BACKGROUND OF THE INVENTION

Angiogenesis, the process of the formation of new blood vessels from pre-existing capillaries, is tightly regulated and normally does not occur except during development, wound healing, and the formation of the corpus luteum during the female reproductive cycle. This strict regulation is manifested by a balanced production of positive and negative factors, which keep angiogenesis in check. However, this balance becomes abrogated under various pathological conditions, such as cancer, diabetes, age-related macular degeneration and retinopathy of prematurity (ROP), resulting in the growth of new blood vessels. It is now well accepted that the progressive growth and metastasis of many solid tumors and loss of vision with diabetes are dependent on the growth of new blood vessels.

One category of diseases that has a neovascular component is vascular diseases of the eye, and tumors of the central nervous system, such as retinoblastoma and primitive neuroectodermal tumors (PNETs). The eye is generally considered to be an extension of the brain, and therefore a part of the central nervous system. PNETs and ocular vascular diseases share similar pathogenesis, specifically, all have a choroidal neovascularization and/or retinal neovascularization component.

Vascular diseases of the eye comprise a major cause of blindness. These diseases include various retinopathies and macular degeneration. Existing treatments include laser ablation of various regions of the retina; vitrectomy or removal of the cloudy vitreous humor and its replacement with a saline solution; and administration of antioxidant vitamins E and C, but none of these methods can cure the disease. Further, existing invasive treatment methods often result in significant loss of vision. Non-invasive methods of treatment are experimental and have not been shown to substantially reduce the risk of blindness or loss of sight.

The anti-tumor activity of vitamin D compounds has been demonstrated in preclinical and/or clinical tests against a variety of cancers, including retinoblastoma. However, the molecular and cellular mechanisms responsible for tumor growth inhibition have not been understood. Past studies examining the effect of calcitriol on retinoblastoma (see, Albert et al. Invest Opthalmol Vis Sci. 1992 July; 33(8):2354-64) have given no indication that VD's effect on retinoblastoma can be generalized to inhibiting neovascular growth in the eye.

Similarly, although there have been suggestions that calcitriol may potentially have antiangiogenic activity (Mantell et al. 1 alpha,25-Dihydroxyvitamin D(3) inhibits angiogenesis in vitro and in vivo. Circ Res. 2000; 87:214-220; Shokravi et al. Vitamin D inhibits angiogenesis in transgenic murine retinoblastoma. Invest. Opthalmol. Vis. Sci. 1995; 36:83-87), there has never been any demonstration that calcitriol is an inhibitor of retinal neovascularization or inhibits retinal EC capillary morphogenesis.

Therefore, there is great interest in the development that can inhibit angiogenesis as a means of treating a variety of diseases with a neovascular or angiogenic component, especially eye diseases with a neovascularization component.

there is a need for more effective methods of treatment of ocular vascular diseases and PNETs.

### SUMMARY OF THE INVENTION

It has been now surprisingly discovered that Vitamin D or a derivative or analogue thereof (VD), is a potent inhibitor of retinal or choroidal endothelial cell (EC) capillary morphogenesis and retinal neovascularization. Accordingly, the present invention disclose compositions and methods for using VD for treating a variety of eye diseases with a neovascularization component.

In one embodiment, the present invention provides a method of treating pathological conditions resulting from ocular angiogenesis and neovascular growth of the eye comprising administration of an effective amount of VD, in particular calcitriol. Diseases or conditions treatable using the compositions and methods of the present invention include non-neoplastic eye diseases, certain neoplastic eye diseases, and certain primitive neuroectodermal tumors (PNETs).

Non-neoplastic eye diseases treatable using the presenting invention include ROP, AMD, diabetic retinopathy, hypertensive retinopathy, central retinal vein occlusion (CRVO), branch vein occlusion (BRVO), neovascular glaucoma, ocular ischemic syndrome, occlusive vasculitis, polypoidal choroidal vasculopathy, myopic choroidal neovascularization, radiation retinopathy, chorioretinitis, central serous choroidopathy, central retinal artery occlusion, uveitic macular edema, idiopathic juxtafoveal telangiectasia, angioid streaks, sickle cell retinopathy, and pseudophakic cystoid macular edema. All of these diseases share the same mechanism of choroidal neovascularization and/or retinal neovascularization, the inhibition of which will result in treatment of the diseases.

Neoplastic eye diseases include primary ocular tumors, such as uveal melanomas, melanocytomas, retinocytomas, retinal hamartomas and choristomas, retinal angiomas, retinal gliomas and astocytomas, choroidal hemangiomas, choroidal neurofibromas, choroidal hamartomas and choristomas, ocular lymphomas and ocular phakomatoses; and metastatic ocular tumors related to choroidal and retinal neovascularization. Similar to the non-neoplastic diseases, the above tumors also share the retinal neovascularization as a key component.

In addition, the present invention is suitable for the treatment PNETs, including PNETs that affect the brain and spinal cord (e.g. medulloblastoma, pineoblastoma, non-pineal supratententorial), and Ewings sarcoma.

In one exemplary embodiment, this invention provides a method of treating neovascular growth in the eye in a subject in need thereof comprising administering an effective amount of calcitriol having a structure represented by Formula I or a salt or prodrug thereof wherein neovascular growth is decreased in the eye.

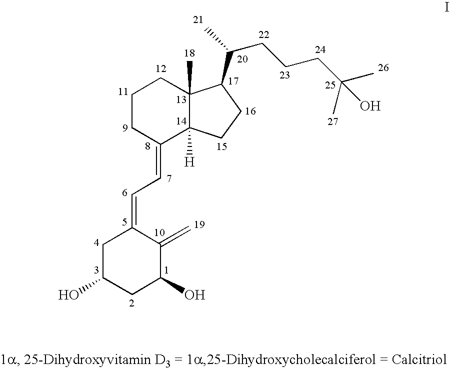

In another exemplary embodiment, this invention provides methods for using a composition suitable for treating angiogenesis and/or neovascular growth in the eye. The composition comprises a compound, prodrug or salt of Formula I; and a pharmaceutically acceptable carrier or excipient. Preferably, in the composition, the first ingredient is calcitriol.

In exemplary embodiments, the invention relates to a method of treating, in a subject in need thereof, non-neoplastic neovascular growth, such as, various retinopathies of the eye and dermatological vasculopathies, e.g. vascular birthmarks. The method comprises administering an effective amount of calcitriol having a structure represented by Formula I or a salt or prodrug thereof wherein the non-neoplastic neovascular growth is decreased.

In various exemplary embodiments, the neovascular forming condition to be treated is diabetic retinopathy, retinopathy of prematurity, hypertensive retinopathy or macular degeneration.

In various exemplary embodiments, the neovascular forming conditions to be inhibited are dermatological vasculopathies, diabetic retinopathy, retinopathy of prematurity, hypertensive retinopathy and macular degeneration.

In another exemplary embodiment, this invention provides methods for using a composition suitable for inhibiting angiogenesis and/or neovascular growth. The composition comprises an analogue, homologue, or derivative of Vitamin D, such as: a compound, prodrug or salt of Formula I; and a pharmaceutically acceptable carrier.

These and other features and advantages of various exemplary embodiments of the methods according to this invention are described in, or are apparent from, the following detailed description of various exemplary embodiments of the methods according to this invention.

### BRIEF DESCRIPTION OF THE FIGURES

FIGS. 1A-1E illustrate an assessment of retinal vasculature in control (FIG. 1A and FIG. 1C) and calcitriol treated (FIG. 1B and FIG. 1D) mice during oxygen-induced ischemic retinopathy (OIR). FIGS. 1A-1D are wholemounts showing immunohistochemical staining of retinal preparation in control vs. treated groups. FIG. 1E is a histogram illustrating that the effect of calcitriol on inhibiting neovascular growth in the retina is dose dependent. The difference in the degree of neovascularization between control and calcitriol-treated mice is significant (P<0.001, for all 3 groups). These experiments were repeated three times with similar results (FIGS. 1A and 1B: bar=500 μm; FIGS. 1C and 1D: bar=50 μm).

FIGS. 2A and 2B illustrate an assessment of vascular endothelial growth factor (VEGF) levels in eyes from control and calcitriol-treated mice. FIG. 2A is a Western blot of VEGF and β-catenin in control and calcitriol treated animals. FIG. 2B is a quantitiative assessment of relative band intensities of the Western blots shown in FIG. 2A. Data in each column are the mean ±SD values of relative intensities of three experiments. Note that there is no significant difference in the relative amounts of VEGF expressed in control and calcitriol-treated eyes (P L 0.5).

FIGS. 3A and 3B illustrate the effects of calcitriol treatment on body weight. Body weights of control (FIG. 3A) and calcitriol-treated (5 μg/Kg), FIG. 3B) mice during oxygen-induced ischemic retinopathy were determined at P12 (before treatment) and at P17 (after treatment). Data in each bar are the mean values of body weights of 4 mice from 4 experiments; Bars; Mean ±SD. There was significant weight gain in control mice from P12 to P17, while calcitriol-treated mice failed to gain weight (P<0.05). A similar lack of weight gain was observed in mice treated with the lower doses of calcitriol (0.5 and 2.5 μg/Kg).

FIG. 4 illustrates the effects of calcitriol on retinal endothelial cell proliferation. Retinal endothelial cells were incubated with different concentrations of calcitriol for 3 days. The degree of cell proliferation relative to the control treatment was determined using a nonradioactive cell proliferation assay as described below. Data are plotted as optical density (OD) vs. μM calcitriol dose. Calcitriol had no effect on endothelial cell proliferation at concentrations below 10 μM, and at 100 μM inhibited cell proliferation by 90%. These experiments were repeated four times with similar results.

FIGS. 5A-C show the effects of calcitriol on retinal EC migration and morphogenesis. Retinal EC migration in the presence of ethanol (control) or calcitriol (10 μM) was determined using wound migration (FIG. 5A and FIG. 5B) and transwell (FIG. 5C) assays as described below. Data in each bar are the mean ±SD values of cells that migrated through the membrane in 10 high-power fields of three separate experiments. Note that there is no significant difference in the degree of migration among control and calcitriol-treated cells (P<0.5).

FIGS. 6A through 6D illustrate the effect of calcitriol on retinal endothelial cell capillary morphogenesis in Matrigel™. The ability of retinal endothelial cell to undergo capillary morphogenesis in the presence of solvent control (FIG. 6A) and calcitriol (10 μM) (FIG. 6B) in Matrigel™ was determined as described in above. Images were obtained after 18 h. Calcitriol diminished the ability of retinal endothelial cells to undergo capillary morphogenesis to the point that no capillaries are observed in FIG. 6B. This concentration of calcitriol had no significant effect on the proliferation of retinal endothelial cells. These experiments were repeated three times with similar results. Bar=40 μm. FIGS. 6C and 6D are higher magnifications (×100) of FIGS. 6A and 6B (×40) respectively.

### DETAILED DESCRIPTION OF EXEMPLARY EMBODIMENTS

As indicated above, the present inventors surprisingly discovered that Vitamin D, or a metabolite, a derivative or analogue thereof (VD), potently inhibits retinal endothelial cell (EC) capillary morphogenesis, and retinal neovascularization. Accordingly, the present invention disclose methods of using VD for treating a variety of diseases with a neovascularization component (hereinafter collectively referred to as “neovascularization diseases”), the method of the present invention comprising administration of a suitable pharmaceutical composition comprising an amount of VD effective for inhibiting neovascularization to a subject in need thereof.

In one embodiment, the present invention provides a method of treating pathological conditions resulting from angiogenesis or neovascular growth of the eye comprising administration of an effective amount of VD, in particular calcitriol. Diseases or conditions treatable using the compositions and methods of the present invention include non-neoplastic eye diseases, certain neoplastic eye diseases, and certain primitive neuroectodermal tumors (PNETs).

Non-neoplastic eye diseases treatable using the presenting invention include ROP, AMD, diabetic retinopathy, hypertensive retinopathy, central retinal vein occlusion (CRVO), branch vein occlusion (BRVO), neovascular glaucoma, ocular ischemic syndrome, occlusive vasculitis, polypoidal choroidal vasculopathy, myopic choroidal neovascularization, radiation retinopathy, chorioretinitis, central serous choroidopathy, central retinal artery occlusion, uveitic macular edema, idiopathic juxtafoveal telangiectasia, angioid streaks, sickle cell retinopathy, and pseudophakic cystoid macular edema. All of these diseases share the same mechanism of choroidal neovascularization and/or retinal neovascularization, the inhibition of which will result in treatment of the diseases.

Neoplastic eye diseases include primary ocular tumors, such as uveal melanomas, melanocytomas, retinocytomas, retinal hamartomas and choristomas, retinal angiomas, retinal gliomas and astocytomas, choroidal hemangiomas, choroidal neurofibromas, choroidal hamartomas and choristomas, ocular lymphomas and ocular phakomatoses; and metastatic ocular tumors related to choroidal and retinal neovascularization. Similar to the non-neoplastic diseases, the above tumors also share the retinal neovascularization as a key component.

In addition, the present invention is suitable for the treatment PNETs, including PNETs that affect the brain and spinal cord (e.g. medulloblastoma, pineoblastoma, non-pineal supratententorial), and Ewings sarcoma.

VD is a group of fat-soluble prohormones, two major forms of which are vitamin D2 (ergocalciferol) and D3 (cholecalciferol). Active vitamin D's include those carrying a hydroxyl group on one or both of the C1 position on the sterol A ring and the C25 position on the side chain, for example, calcitriol (1α,25-dihydroxy vitamin D), 1α,24-dihydroxy vitamin D, α-calcidol (1α-monohydroxy vitamin D), calcifedol (25-monohydroxy vitamin D), 1α,24,25-trihydroxy vitamin D, 1β,25-dihydroxy vitamin D, 22-oxacalcitriol, and calcipotriol. Analogues thereof also include dihydrotachysterol. Many others analogs of vitamin D are known in the art that are generated by a variety of chemical modifications. These modifications are made to improve the potency of vitamin D in different applications while reducing its potential toxic side effects. One of ordinary skills in the art readily recognize that any Vitamin D or its analogue, metabolite, or derivative is a suitable for the present invention. See also those disclosed in e.g. U.S. Pat. Nos. 4,749,710, 6,806,262, and 7,211,680.

As used herein, “administering” or “administration” includes any means for introducing a substance into the body, preferably into the systemic circulation. Examples include but are not limited to oral; buccal, sublingual, pulmonary, transdermal, transmucosal, as well as subcutaneous, intraperitoneal, intravenous, and intramuscular injection.

A “therapeutically effective amount” means an amount of a substance that, when administered to a subject for treating a disease, is sufficient to effect such treatment for the disease. The “therapeutically effective amount” will vary depending on the compound, the disease state being treated, the severity or the disease treated, the age and relative health of the subject, the route and form of administration, the judgment of the attending medical or veterinary practitioner, and other factors.

For purposes of the present invention, “treating” or “treatment” describes the management and care of a patient for the purpose of combating the disease, condition, or disorder. The terms embrace both preventative, i.e., prophylactic, and palliative treatment. Treating includes the administration of a compound of present invention to prevent the onset of the symptoms or complications, alleviating the symptoms or complications, or eliminating the disease, condition, or disorder.

The pharmaceutical preparations administerable by the invention can be prepared by known dissolving, mixing, granulating, or tablet-forming processes. For oral administration, the anti-infective compounds or their physiologically tolerated derivatives such as salts, esters, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

As used herein, “pharmaceutical composition” means therapeutically effective amounts of the anti-neovascular compound together with suitable diluents, preservatives, solubilizers, emulsifiers, and adjuvants, collectively “pharmaceutically-acceptable carriers.” As used herein, the terms “effective amount” and “therapeutically effective amount” refer to the quantity of active therapeutic agent sufficient to yield a desired therapeutic response without undue adverse side effects such as toxicity, irritation, or allergic response. The specific “effective amount” will, obviously, vary with such factors as the particular condition being treated, the physical condition of the subject, the type of animal being treated, the duration of the treatment, the nature of concurrent therapy (if any), and the specific formulations employed and the structure of the compounds or its derivatives. In this case, an amount would be deemed therapeutically effective if it resulted in one or more of the following: (a) inhibition of neovascular growth; and (b) the reversal or stabilization of occular neovascular growth. The optimum effective amounts can be readily determined by one of ordinary skill in the art using routine experimentation.

Pharmaceutical compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The preparation of pharmaceutical compositions which contain an active component is well understood in the art. Such compositions may be prepared as aerosols delivered to the nasopharynx or as injectables, either as liquid solutions or suspensions; however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like or any combination thereof.

In addition, the composition can contain minor amounts of auxiliary substances such as wetting or emulsifying agents, pH buffering agents which enhance the effectiveness of the active ingredient.

The methods of administering an effective dose of the antiangiogenic composition of calcitriol according to the invention includes pharmaceutical preparations comprising the anti-angiogenic compound alone, or can further include a pharmaceutically acceptable carrier, and can be in solid or liquid form such as tablets, powders, capsules, pellets, solutions, suspensions, elixirs, emulsions, gels, creams, or suppositories, including rectal and urethral suppositories. Pharmaceutically acceptable carriers include gums, starches, sugars, cellulosic materials, and mixtures thereof. The pharmaceutical preparation containing the anti-infective compound can be administered to a subject by, for example, subcutaneous implantation of a pellet. In a further embodiment, a pellet provides for controlled release of anti-infective compound over a period of time. The preparation can also be administered by intravenous, intraarterial, or intramuscular injection of a liquid preparation oral administration of a liquid or solid preparation, or by topical application. Administration can also be accomplished by use of a rectal suppository or a urethral suppository.

Examples of suitable oily vehicles or solvents are vegetable or animal oils such as sunflower oil or fish-liver oil. Preparations can be effected both as dry and as wet granules. For parenteral administration (subcutaneous, intravenous, intraararterial, or intramuscular injection), the anti-neovascular compounds or its physiologically tolerated derivatives such as salts, esters, N-oxides, and the like are converted into a solution, suspension or expulsion, if desired with the substances customary and suitable for this purpose for example, solubilizers or other auxiliaries. Examples are sterile liquids such as oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, preferred liquid carriers are oils with particularly exemplary embodiments being vegetable oil and the like.

Pharmaceutically acceptable carriers for controlled or sustained release compositions administerable according to the invention include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

It should be noted that, while the investigations described herein use a post-mortem histo-chemical examination of the mouse eye to determine the extent of visualization, there are a variety of methods known in the art to assess the visual acuity and/or pathology of the eye. For example, a “tangent screen” or “Goldmann perimeter” test effectively measures the size of the subjects visual field by moving an object or a light from the periphery of the field toward the center. Such examinations allow the identification of blind spots. Such examinations now also include computerized automated perimetry. Macular degeneration can be home-monitored by tests using an Amsler grid, which comprises a black card having a white grid with a white dot in its center. The subject then looks at the grid with one eye noting any distortion in the grid-lines. Unviewable areas indicate a blind spot while the appearance of wavy lines indicates there may be a vision problem.

More objective examinations of the eye may be made by a trained professional using various methods of ophthalmascopy. Opthalmoscopy allows the physician to see into the eye using several types of instruments. Such instruments include, a direct opthalmoscope, which is an instrument resembling a small flashlight with several lenses that can magnify the fundus or back of the eye by about 15 times; an indirect opthalmoscope is an instrument resembling a miner's lamp that is worn about the head. While an indirect opthalmoscope magnifies only 3 to 5 times it allows a wider angle of view with a better view of the fundus. A slit lamp is a binocular device having a narrow beam focused on the fundus and viewed through a microscope. This instrument provides greater magnification but a smaller field of view and is mainly used to view the center of the fundus and the optic nerve. Other, more quantitative methods include fluorescein angiography which allows clear visualization of the retinal blood vessels using a fluorescent dye visualized by a series of photographs.

Previous research on the effects of calcitriol on angiogenesis have been inconclusive. Calcitriol has been reported to decrease (Merke et al., Identification and regulation of 1,25-dihydroxyvitamin D₃ receptor activity and biosynthesis of 1,25-dihydroxyvitamin D₃: studies in culture bovine aortic endothelial cells and human dermal capillaries. J Clin Invest. 1989; 83:1903-1915) or have no effect (Wang D S, et al., Anabolic effects of 1,25-dihydroxyvitamin D₃ on osteoblasts are enhanced by vascular endothelial growth factor produced by osteoblasts and by growth factors produced by endothelial cells. Endocrinology. 1997; 138:2953-2962) on endothelial cell proliferation; to have no effect on capillary morphogenesis in vitro (Lansink M, et al., Effects of steroid hormones and retinoids on the formation of capillary-like tubular structures of human microvascular endothelial cells in fibrin matrices is related to urokinase expression. Blood. 1998; 92:927-938.); and to inhibit angiogenesis in vivo (Oikawa T, et al. Inhibition of angiogenesis by vitamin D₃ analogues. Eur J. Pharmacol. 1990; 178:247-250). Further, studies on the effect of vitamin D on retinoblastomas showed two contraindications to its use in ocular diseases.

Mantell et al. (2000) (1α,25-dihydroxyvitamin D₃ inhibits angiogenesis in vitro and in vivo. Circ. Res. 2000; 87:214-220) suggested that VD may have a potential antiangiogenic activity in some tumors, but statistical analysis showed that the effect was not significant. In these studies, nude mice were injected with MCF-7 breast carcinoma cell that had been induced to overexpress VEGF and MDA-435S breast carcinoma cells. Further, while there appeared to be a decrease in vascularization of the tumors, there was no significant decrease in tumor size nor was there a difference in the proportion of MCF₇ and MDA-435S cells present in the tumor. In any event, these studies do not provide any teaching or suggestion that VD would have inhibitive effects against retinal or choroidal vascularization.

Similarly, although it was known that vitamin D has certain effects in arresting retinoblastoma growth, this effect appeared to be related to the presence of a high affinity receptor specific for calcitriol, leading to the conclusion that inhibition by vitamin D of retinoblastoma growth is proportionate to the quantity and affinity of the vitamin D receptor of each particular cell type. There was no teaching or suggestion that VD's effect on retinoblastoma may in any way be generalized to other type of diseases.

This invention discloses for the first time that VD inhibits retinal neovascularization, retinal endothelial cell proliferation, and capillary morphogenesis. In the investigations described herein the inventors show that calcitriol significantly blocked retinal neovascularization during OIR at doses shown to be effective in inhibition of retinoblastoma with minimal toxicity. The effects of calcitriol on inhibition of angiogenesis were independent of changes in VEGF expression. To the inventors knowledge these investigations show that calcitriol is one of the most potent inhibitors of angiogenesis in the OIR model.

The inventors' results indicate that the anti-angiogenesis effect of VD is not dependent on the inhibition of EC proliferation. No significant inhibition of retinal endothelial cell proliferation was observed when calcitriol was used up to 10 μM, but completely abolished the ability of retinal endothelial cell to undergo capillary morphogenesis. This concentration had no effect on retinal endothelial cell proliferation in short (3 days) or long (9 days) incubation with calcitriol. This is consistent with the in vivo data showing that retinal neovascularization was dramatically inhibited in the presence of chemotherapeutic doses of calcitriol.

At higher concentrations (e.g. 50 μM), significant inhibition of retinal endothelial cell proliferation was observed. At 100 μM, calcitriol inhibited retinal endothelial cell proliferation by 90%. These inhibitory concentrations are much higher than those used in many studies that reported no or mild effects on endothelial cell proliferation. Therefore, inhibition of endothelial cell proliferation may require higher concentrations of calcitriol. However, calcitriol at 10 μM completely abolished the ability of retinal endothelial cell to undergo capillary morphogenesis.

### EXAMPLES

### Materials and Methods

Calcitriol

Pure crystalline calcitriol (provided by ILEX Oncology Inc., San Antonio, Tex.) was prepared for injection as previously described (Albert D M, et al., Vitamin D analogs, a new treatment for retinoblastoma: the first Ellsworth lecture. Ophthamic Genet. 2002; 23:137-156). Briefly, the crystalline calcitriol was dissolved in 100% ethanol for a stock solution of 1 mg/ml and stored in amber bottles under argon gas at −70° C. The stock solution was diluted in mineral oil to a concentrations of 0.0025, 0.0125 and 0.025 μg/0.1 ml. Each mouse in the treatment group received 0.0025, 0.0125 or 0.025 μg of calcitriol (approximately 0.5, 2.5 and 5 μg/Kg) per treatment. These doses were previously found (Albert D M, et al., Vitamin D analogs, a new treatment for retinoblastoma: the first Ellsworth lecture. Ophthamic Genet. 2002; 23:137-156, Sabet S J, et al., Antineoplastic effect and toxicity of 1,25-dihydroxy-16-ene-23-yne-vitamin D₃ in athymic mice with Y-79 human retinoblastoma tumors. Arch Opthalmol. 1999; 117:365-370) to be an effective dose with minimal toxicity. For in vitro studies, a stock solution of calcitriol in 100% ethanol (2 mM, 0.83 mg/ml) was prepared.

Mouse Model of Oxygen-Induced Ischemic Retinopathy

All experimental procedures involving animals were performed according to the ARVO Statement for the Use of Animals in Ophthalmic and Vision Research. The mouse oxygen-induced ischemic retinopathy (OIR) model (Smith L E, et al. Oxygen-induced retinopathy in the mouse. Invest. Opthalmol. Vis. Sci. 1994; 35:101-111) was used to evaluate the effects of calcitriol on retinal neovascularization. In this model, 7-day-old (P7) pups (8-10 pups) and their mother were placed in an airtight incubator and exposed to an atmosphere of 75±0.5% oxygen (hyperoxia) for 5 days. Incubator temperature was maintained at 23±2° C., and oxygen was continuously monitored with a PROOX model 110 oxygen controller (Reming Bioinstruments Co., Redfield, N.Y.). Mice were then brought to room air for 5 days. Maximum retinal neovascularization occurred from P12 to P17. To assess anti-angiogenic activity of calcitriol, half of the pups were injected intraperitoneally with 0.025 μg calcitriol in 0.1 ml mineral oil per day from P12 to P17. The other half of the littermates was injected with 0.1 ml of mineral oil. Generally one eye from each mouse was used for histochemical analysis and the other eye for histological evaluation as outlined below. These experiments were repeated at least 3 times for each dose.

Calcium Toxicity

A cytotoxic side effect of calcitriol treatment is loss of body weight due to hypercalcemia. The antineoplastic effect of calcitriol, however, is unrelated to either high serum calcium levels or calcium deposition in the tumors. In fact, the clinical usefulness of vitamin D is limited by the toxic effects associated with hypercalcemia. The inventors evaluated the body weights of mice injected with solvent control or calcitriol during OIR. The body weight of mice injected with solvent control from P12 to P17 was increased by 30%, while the body weight of mice injected with calcitriol was decreased by 20%. These are consistent with previous mouse studies and indicate a potential side effect of calcitriol treatment.

Visualization and Quantification of Retinal Neovascularization

Vessel obliteration and retinal vascular pattern were analyzed using retinal wholemounts stained with anti-Collagen IV antibody as previously described (Wang S, et al. Thrombospondin-1-deficient mice exhibit increased vascular density during retinal vascular development and are less sensitive to hyperoxia-mediated vessel obliteration. Dev Dyn. 2003; 228:630-642., Wang S, et al. Attenuation of Retinal Vascular Development and Neovascularization during Oxygen-Induced Ischemic Retinopathy in Bcl-2−/− Mice. Developmental Biol. 2005; 279:205-219). Briefly, P17 mouse eyes were enucleated and briefly fixed in 4% paraformaldehyde in phosphate buffered saline (PBS) (10 min on ice). The paraformaldehyde fixed eyes were then fixed in 70% ethanol for at least 24 h at −20° C. Retinas were dissected in PBS and then washed with PBS 3 times, 10 min each. Following incubation in blocking buffer (50% fetal calf serum, 20% normal goat serum (NGS) in PBS) for 2 h, the retinas were incubated with rabbit anti-mouse collagen IV (Chemicon, diluted 1:500 in PBS containing 20% fetal calf serum, 20% normal goat serum) at 4° C. overnight. Retinas were then washed 3 times with PBS, 10 min each, incubated with a secondary antibody Alexa 594-labeled goat-anti-rabbit (Invitrogen, Carlsbad, Calif.), at 1:500 dilution prepared in PBS containing 20% FCS, 20% NGS for 2 h at room temperature, washed 4 times with PBS (30 min each), and mounted on a slide with PBS/glycerol (2 vol/1 vol). Retinas were viewed by fluorescence microscopy, and images were captured in digital format using a Zeiss microscope (Carl Zeiss, Chester, Va.).

Quantification of Retinal Neovascularization

Quantification of retinal neovascularization on P17 was performed by counting vascular cell nuclei anterior to the in limiting membrane (Wang S, et al., Attenuation of Retinal Vascular Development and Neovascularization during Oxygen-Induced Ischemic Retinopathy in Bcl-2−/− Mice. Developmental Biol. 2005; 279:205-219). Briefly, mice eyes were enucleated, fixed in formalin for 24 h, and embedded in paraffin. Serial sections (6 μm), each separated by at least 40 μm, were taken from around the region of the optic nerve. The hematoxylin- and PAS-stained sections were examined in masked fashion, by two independent observers without the knowledge of the samples identity, the presence of neovascular tufts projecting into the vitreous from the retina. The neovascular score was defined as the mean number of neovascular nuclei per section found in eight sections (four on each side of the optic nerve) per eye; generally four eyes from different mice per experiment were used.

Western Blot Analysis

Vascular endothelial growth factor (VEGF) levels were determined by Western blotting of whole eye extracts prepared from P15 mice during OIR (5 days of hyperoxia and 3 days of normoxia) when maximum levels of VEGF were expressed (Wang S, et al. Attenuation of Retinal Vascular Development and Neovascularization during Oxygen-Induced Ischemic Retinopathy in Bcl-2−/− Mice. Developmental Biol. 2005; 279:205-219). Mice were euthanized by CO₂ inhalation, then eyes from 2 or 3 mice dissected, homogenized in 0.2 ml of RIPA buffer, 10 mM HEPES pH 7.6, 142.5 mM KCl, 1% NP-40, and protease inhibitor cocktail, (Roche Applied Science, Indianapolis, Ind.), sonicated briefly, and incubated at 4° C. for 20 min. The resulting homogenates were then centrifuged at 16,000×g for 10 min at 4° C. to remove insoluble material. Supernatants were transferred to a clean tube, and protein concentrations were determined using the DC Protein Assay (Bio-Rad Laboratories, Hercules, Calif., Cat. No. 500-0111). Approximately 20 μg of protein from the centrifuged homogenates were analyzed by SDS-PAGE (4-20% Tris-Glycin gel, Invitrogen, Carlsbad, Calif.) under reducing conditions and transferred to a nitrocellulose membrane. The blot was incubated with a rabbit polyclonal anti-mouse VEGF antibody (1:2000 dilution; PeproTech, Rock Hill, N.J.), washed, and developed using a goat anti-rabbit HRP-conjugated secondary antibody (1:5000; Jackson Immunoresearch Laboratories, West Grove, Pa.) and ECL system (Amersham Biosciences, Piscataway, N.J.). The same blot was also probed with a monoclonal antibody to β-catenin (1:3000; BD Transduction Laboratories, BD Biosciences, San Jose, Calif.) to verify equal protein loading in all lanes. For quantitative assessments, the band intensities relative to loading controls were determined by scanning the blots using the Molecular Dynamics Storm 860 Scanner and Image Quant Software (Amersham Biosciences, Piscataway, N.J.).

Toxic Effect Assessment

The side effects of calcitriol on the mouse body weights were determined. In the OIR studies, all pups had similar body weight prior to initiation of the experiment (P12) and after exposure to high oxygen (P17). None of the experimental animals died during these experiments.

Determination of Serum Calcium Levels

Blood (0.2 ml) was collected from P17 mice treated with calcitriol or solvent control during OIR. The blood was allowed to clot at room temperature, centrifuged, the serum was transferred to a clean tube, and stored at −80° C. until needed for analysis. Serum samples were sent to Marshfield Clinic (Marshfield, Wis.) for total serum calcium analysis. The serum calcium level is reported as mg/dL.

Retinal EC Proliferation, Migration and Capillary Morphogenesis

Primary mouse retinal endothelial cell (REC) cultures were prepared and maintained as described previously (Su X, et al., Isolation and characterization of murine retinal endothelial cells. Mol. Vision. 2003; 9:171-178). Briefly, REC were isolated from wild type or transgenic-immortomouse by collagenase digestion of retina and affinity purification using magnetic beads coated with platelet/endothelial cell adhesion molecule-1 (anti-PECAM-1). The bound cells were plated on fibronectin-coated wells and expanded. The REC were characterized for expression and localization of endothelial cell markers by fluorescence-activated cell sorting (FACS) analysis and indirect immuno fluorescence staining. The ability of these cells to form capillary like networks was assessed on matrigel™. For cell proliferation assays, retinal endothelial cell (10,000) were plated in triplicate in 96-well plates and incubated overnight. On the following day, cells were fed with growth medium containing various concentrations of calcitriol or solvent control. Cells were allowed to grow for the indicated period of time and were fed every 3 days with fresh medium containing appropriate concentrations of calcitriol. The degree of proliferation was assessed using the nonradioactive cell proliferation assay (CellTiter 96® AQᵤₑₒᵤₛ; Promega, Madison, Wis.) as recommended by the supplier.

Retinal EC migration was determined using both wound migration and transwell assays. Confluent monolayers of retinal EC were wounded using a micropipette tip, rinsed with growth medium to remove detached cells, and incubated with growth medium containing calcitriol (10 μM) or ethanol (solvent control). Wound closure was monitored by phase microscopy, and digital images were obtained at different time points used for quantitative assessment of migration. For transwell migration, wells (8 μm pore size, 6.5 mm membrane; Costar) were coated with Matrigel™ (200 μg/ml) or fibronectin (2 μg/ml) in PBS on the bottom side at 4° C. overnight. The next day, inserts were rinsed with PBS, blocked in PBS containing 2% BSA for 1 h at room temperature, and washed with PBS. Cells were removed by trypsin-EDTA, counted, and resuspended at 1×10⁶ cells/ml in serum-free medium. Inserts were placed in 24-well dishes (Costar) containing 0.5 ml of serum-free medium and 0.1 ml of cell suspension was then added to the top of the insert. Cells were allowed to migrate through the filter for 3 h in a tissue culture incubator. After incubation, the cells on the top of the filter were scraped off using a cotton swab; the membrane was then fixed in 4% paraformaldehyde and stained with hematoxylin and eosin. The inserts were then mounted on a slide cell side up, and the number of cells which migrated to the bottom of the filter was determined by counting 10 high power fields at ×200 magnification.

The ability of the cultured retinal endothelial cells to form capillary like networks was assessed on Matrigel™ (BD Biosciences, San Jose, Calif.). The capillary morphogenesis assays in Matrigel™ were performed as previously described (Su X, et al., Isolation and characterization of murine retinal endothelial cells. Mol. Vision. 2003; 9:171-178; Rothermel T A, et al., Polyoma virus middle-T-transformed PECAM-1 deficient mouse brain endothelial cells proliferate rapidly in culture and form hemangiomas in mice. J Cell Physiol. 2005; 202:230-239). Briefly, 0.5 ml of Matrigel™ was added to a cold 35 mm tissue culture plate and incubated at 37° C. for at least 30 min to allow the Matrigel™ to harden. Retinal endothelial cells were removed by trypsin-EDTA, resuspended at 1.5×10⁵ cells/ml in the growth medium containing calcitriol (10 μM) or solvent control, and incubated on ice for 15 min. Following incubation, 2 ml of cell suspension in the presence of calcitriol or solvent control was gently added to the Matrigel™-coated plates and incubated at 37° C. Cultures were monitored for 6-48 h, and images were captured in digital format after 18 h when maximum organization was observed. Longer incubation did not result in further organization of endothelial cells into tubular network. The capillary network formed by control cells began to fall apart at 24-48 h.

Statistical Analysis

Statistical differences between groups were evaluated with Student's unpaired t-test (two-tailed). Mean ±standard deviation is shown. P values≦0.05 were considered significant.

### Example 1 Effects of Calcitriol on Retinal Neovascularization

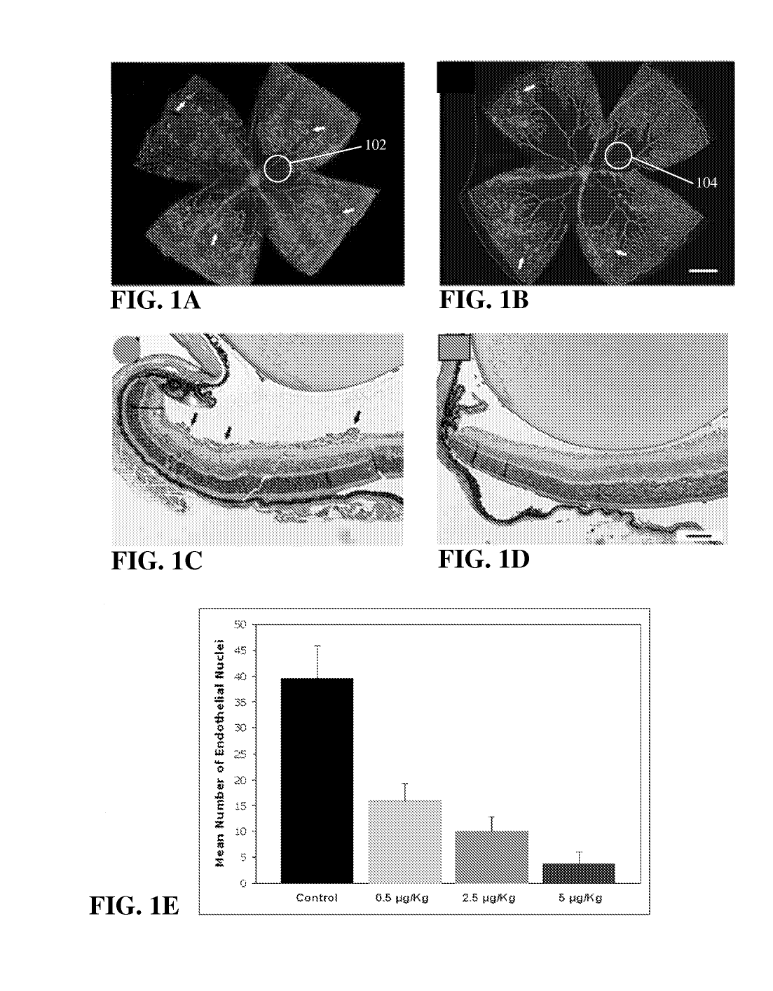

As described previously, the inventors, in performing experiments on the effects of various vitamin D analogs in treating retinoblastoma, included calcitriol as a control. Collagen IV immunohistochemical staining of the wholemount retinas was performed to visualize ischemia-induced retinal neovascularization. In this experiment, P7 mice were exposed to a cycle of hyperoxia and normoxia, and eyes were removed for appropriate analysis as described above. FIGS. 1A and 1B show retinal wholemounts in which the retinal vasculature was visualized by immunohistochemical staining using an anti-collagen IV antibody from P17 control and calcitriol-treated mice exposed to OIR, respectively. FIGS. 1C and 1D show hematoxylin-and periodic acid-Schiff (PAS)-stained cross sections prepared from P17 control and calcitriol-treated mice (0.5 μg/Kg, 2.5 μg/Kg and 5 μg/Kg) exposed to OIR, respectively. Arrows show the new vessels growing into the vitreous compartment. The quantitative assessments of retinal neovascularization in eyes from P17 control and calcitriol-treated mice exposed to OIR are shown in FIG. 1E. Data in each bar are the mean values from 4 eyes of 4 mice; Bars; Mean ±SD. The difference in the degree of neovascularization between control and calcitriol-treated mice is significant (P<0.001, for all 3 groups). These experiments were repeated three times with similar results (FIGS. 1A and 1B: bar=500 μm; FIGS. 1C and 1D: bar=50 μm).

As shown, calcitriol-treated and control P17 mice subjected to OIR demonstrated significant obliteration of the peripapillary retinal capillaries, whereas the larger, well-developed radial retinal vessels extending from the optic disc still existed in areas 102 and 104 shown in FIGS. 1A and 1B. Retinas from P17 control mice exposed to OIR contained many neovascular tufts extending from the surface of the retina at the junction between the perfused and nonperfused retina (arrows, FIG. 1A). In contrast, retinas from P17 mice treated with calcitriol demonstrated markedly reduced neovascularization (arrows, FIG. 1B). These results show that retinal neovascularization in the treated mice was inhibited by greater than 90% at 5 μg/Kg of calcitriol as shown in FIG. 5E (P<0.001). A lower degree of inhibition was observed at lower doses of calcitriol. A 75% inhibition of neovascularization was observed at 2.5 μg/Kg of calcitriol, while 60% inhibition was observed at 0.5 μg/Kg of calcitriol. These data show that the inhibition of angiogenesis by calcitriol is a dose dependent response, highlighting the finding that the decrease in vascularization is an effect of calcitriol not a secondary response to increased serum calcium levels.

### Example 2 Inhibition of Retinal Neovascularization by Calcitriol

Retinas from P17 control mice subjected to OIR contained multiple neovascular tufts on their surface (arrows, FIG. 1C), with some extending into the vitreous. Retinas from mice treated with calcitriol showed significantly fewer preretinal neovascular tufts, P<0.001 (FIG. 1D). The neovascular tufts contained a significant number of neovascular nuclei anterior to the ILM as illustrated by the data shown in Table 1 and FIG. 1E. This data shows that in OIR mice treated with calcitriol at doses of 0.025 μg, retinal neovascularization was inhibited by greater than 90% when compared to the control mice.

TABLE 1
MEAN NUMBER OF ENDOTHELIAL NUCLEI (P < 0.001)

| | |
|---|---|
| CONTROL (n = 4) | 39.9 ± 6.4 (SD) |
| CALCITRIOL (n = 4) 0.025 μg (˜5 μg/Kg) | 3.8 ± 2.2 (SD) |

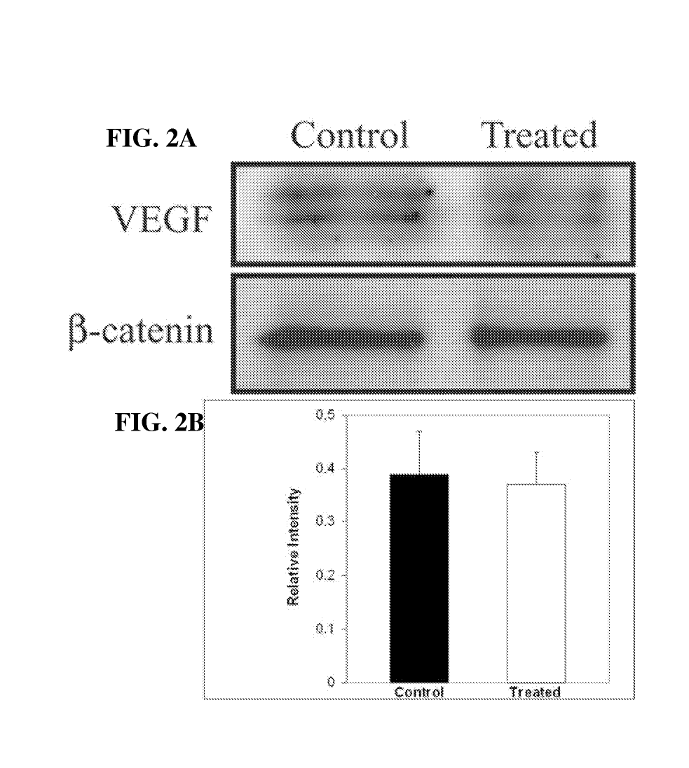

To determine whether the inability of retinas from calcitriol-treated mice to undergo neovascularization in response to ischemia was due to lack of VEGF expression Western blots were performed on the experimental animals. VEGF levels were examined in retinas from P15 control and calcitriol-treated mice during OIR (5 days of hyperoxia and 3 days of normoxia). It has been reported that VEGF expression is maximally induced at P15 during OIR (18). Briefly, eye extracts prepared from control and calcitriol-treated (5 μg/Kg) P15 mice (5 days of hyperoxia and 3 days of normoxia) were analyzed by SDS-PAGE and Western blotting with β-catenin used for loading control (FIG. 2A). The quantitative assessments of relative band intensities are shown in (FIG. 2B). Data in each bar are the mean values of relative intensities of three experiments; Bars; Mean ±SD. There was no significant difference in the relative amounts of VEGF expressed in control and calcitriol treated eyes (P<0.56). FIG. 2A shows a Western blot of protein prepared from whole eye extracts of control and calcitriol-treated P15 mice during OIR. The levels of VEGF expression and in eyes from control and calcitriol-treated mice during OIR were not significantly different P<0.56 (FIG. 2B). These data are provided in Table 2. The lack of any difference in VEGF expression between the treatment groups indicates that the effect of calcitriol on retinal neovascularization is not a result of differential VEGF expression but must result from some other mechanism. Further, the similarity in catenin expression indicates that the difference in retinal endothelial cell response is not due to an overall effect on protein expression but suggests that there is some more specific effect of calcitriol on neovascular growth.

TABLE 2
Relative Intensity of Expressed VEGF in Treatment Groups

| | |
|---|---|
| CONTROL (n = 3) | 0.39 ± 0.08 (SD) |
| CALCITRIOL (n = 3) 0.025 μg (˜5 μg/Kg) | 0.37 ± 0.06 (SD) |

### Example 3 Assessment of Side Effects of Calcitriol on the Body weight

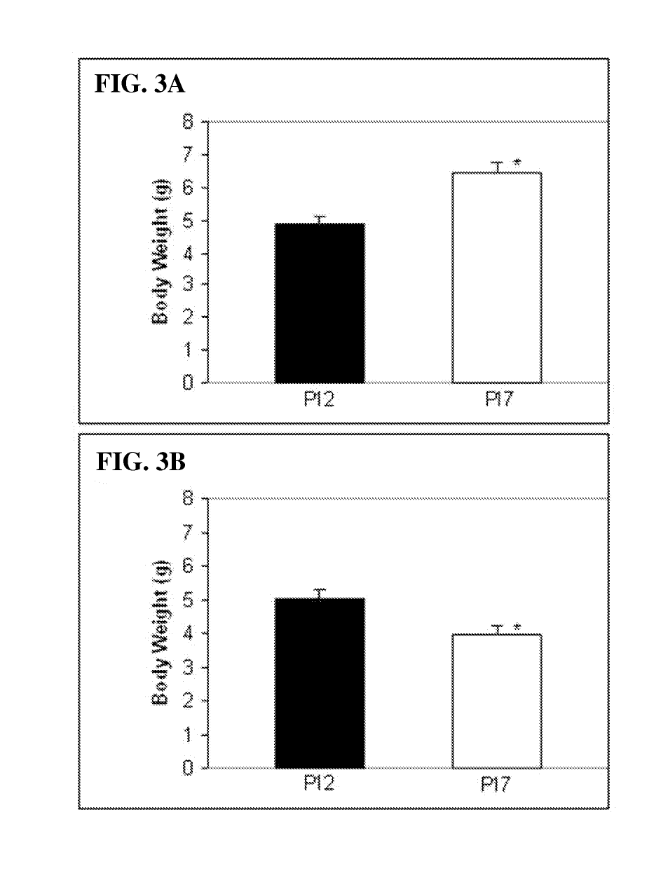

The body weights of experimental animals were determined at P12 and P17 after five days of injection with calcitriol or solvent control. In control mice, there was a significant increase in body weight of about 30% from P12 to P17 (FIG. 3A). In contrast, there was a significant decrease (20%) in the body weights of mice treated with calcitriol for 5 days (FIG. 3B; P<0.05). Thus, mice treated with calcitriol exhibit reduced bodyweights compared to control mice, a common side effect of calcitriol and hypercalcimia (Sabet S J, et al., Antineoplastic effect and toxicity of 1,25-dihydroxy-16-ene-23-yne-vitamin D₃ in athymic mice with Y-79 human retinoblastoma tumors. Arch Opthalmol. 1999; 117:365-370, Dawson D G, et al., Toxicity and dose-response studies of 1α-hydroxyvitamin D₂ in LHβ-Tag transgenic mice. Opthalmology. 2003; 110:835-839), this data is shown in Table 3.

TABLE 3
Change in Body Weight of Treatment Groups

| | P12 | P17 |
|---|---|---|
| Control (n = 4) | 4.85 ± 0.25 | 6.47 ± 0.29 |
| Calcitriol (n = 4) | 5.04 ± 0.23 | 3.93 ± 0.29 |

### Example 4 Calcitriol Inhibits Retinal Endothelial Cell Proliferation and Capillary Morphogenesis in Matrigel™

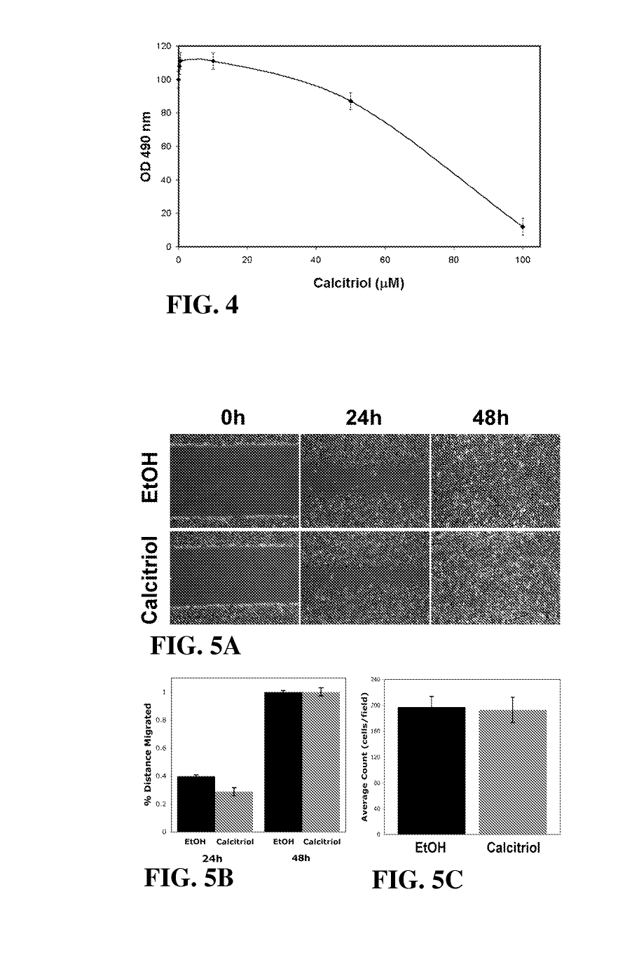

The effects of calcitriol on retinal endothelial cell proliferation have not been previously examined. Furthermore, the effects of calcitriol on proliferation of other types of endothelial cells have been contradictory. The inventors examined the effects of calcitriol on retinal endothelial cell proliferation, with both short-term (3 days) and long-term (9 days) incubation. Table 4 shows the proliferation of retinal endothelial cell incubated with (0 to 100 μM) concentrations of calcitriol relative to cells incubated with solvent control for 3 days at 37° C. Minimal toxicity was observed at lower concentrations of calcitriol (0-10 μM), and, in fact, low doses of calcitriol appear to result in an increase in cell proliferation when standardized to the control group. Significant toxicity was only observed at 50 μM calcitriol and higher. Calcitriol at 100 μM inhibited retinal endothelial cell proliferation by approximately 90%. Incubation of retinal endothelial cell with calcitriol (0-10 μM) for 9 days had minimal effects on their proliferation, similar to those observed after 3 days of exposure. This data is also illustrated in FIG. 4.

TABLE 4
Endothelial Cell Proliferation as
a Function of Calcitriol Treatment

| | |
|---|---|
| Calcitriol (μM) | Relative Survival |

| | |
|---|---|
| 0.00 | 100% |
| 0.25 | 108% |
| 0.50 | 111% |
| 10.00 | 111% |
| 50.00 | 87% |
| 100.00 | 12% |

### Example 5 Calcitriol Inhibits Retinal Endothelial Cell Migration

The effects of calcitriol on cell proliferation showing a biphasic response, the inventors then investigated the effects of calcitriol on retinal EC migration. FIG. 5A shows the effect of-retinal EC migration in the presence of ethanol (control) or calcitriol (10 μM) as determined using wound migration and measured at 0, 24 and 48 hours after administration. The morphology of confluent monolayers of retinal EC wound closure was monitored by phase microscopy at different times post wounding and is shown in FIG. 5A. As shown in FIG. 5A there is little difference between the calcitriol and the control groups. FIG. 5B is a histogram illustrating the quantitative assessment of the two groups. A Student's unpaired t-test shows that the difference between the two groups is not significant.

FIG. 5C illustrate is a quantification of the transwell assay as described above. Briefly, wells were coated with Matrigel™ (200 μg/ml) or fibronectin (2 μg/ml) in PBS on the bottom side at 4° C. overnight. The next day inserts were rinsed with PBS, blocked in PBS containing 2% BSA for 1 h at room temperature and washed with PBS. Cells were removed by trypsin-EDTA, counted, and resuspended at 1×10⁶ cells/ml in serum-free medium. Inserts were placed in 24-well dishes (costar) containing 0.5 ml of serum-free medium, and 0.1 ml of cell suspension was then added to the top of the insert. Cells were allowed to migrate through the filter for 3 h in a tissue culture incubator. After incubation, the cells on the top of the filter were scraped off using a cotton swab. The membrane was fixed in 4% paraformaldehyde and stained with hematoxylin and eosin. The inserts were then mounted on a slide cell side up and the number of cells which migrated to the bottom of the filter was determined by counting 10 high power fields at ×200 magnification. Quantification of this assay (FIG. 5C) shows that there is no difference between the control group and the calcitriol treated group. Using the transwell assay, calcitriol had no significant effect on migration of retinal EC through the filter coated with Matrigel™ (FIG. 5C). However, calcitriol slightly enhanced retinal EC migration through filters coated with fibronectin compared to solvent control (not shown). Therefore, calcitriol at 10 μM had minimal effects on retinal EC migration in culture. For FIG. 5B, data in each bar are the mean values of percent distance migrated from three separate experiments; Bars, Mean ±SD. For FIG. 5C, data in each bar are the mean values of cells migrated through the membrane in 10 high power fields of three separate experiments; Bars, Mean ±SD. Note there is no significant difference in the degree of migration among control and calcitriol-treated cells (P<0.5)

### Example 6 Effects of Calcitriol on Retinal EC Capillary Morphogenesis in Matrigel™

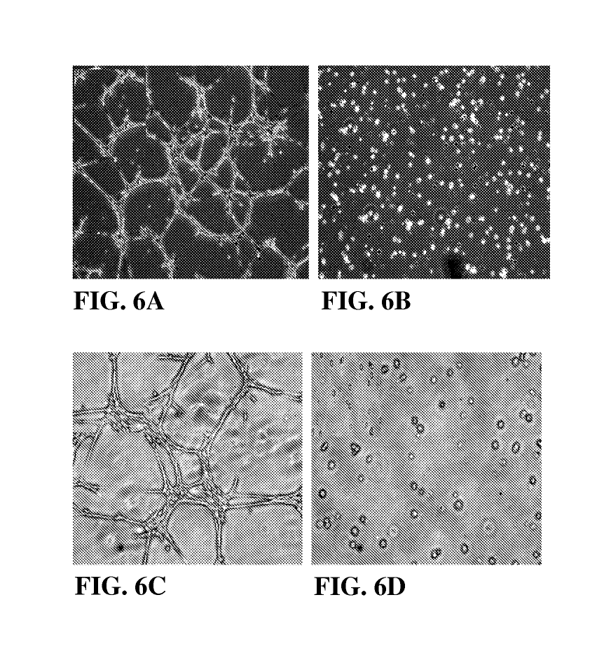

Because there was no decrease in the proliferation or migration of retinal EC cells treated with calcitriol, the inventors then investigated the ability of retinal EC cells to organize into capillary networks. Previous studies have shown that retinal endothelial cells, like many other types of endothelial cells, rapidly organize into capillary networks when plated in Matrigel™. In contrast to the proliferation assays, the morphogenesis assay reveals that, in vitro, even the presence of 10 μM calcitriol inhibits the ability of retinal EC cells to form capillary networks. FIGS. 6A and 6B are 40× magnifications of EC cells cultured on Matrigel™ without calcitriol (6A) and in the presence of 10 μM calcitriol. FIGS. 6C and 6D are the same preparations but at higher magnification (100×). As is shown, in the presence of 10 μM calcitriol capillary morphogenesis was completely inhibited. This concentration of calcitriol, as shown in Table 4, results in an increase in EC cell proliferation yet, as disclosed herein, results in a complete absence of capillary formation. This in vitro data is consistent with the in vivo data which shows the inhibition of retinal neovascularization by calcitriol as illustrated in FIGS. 1A and 1B and discussed above.

The inventors have shown that calcitriol, in vivo, inhibits retinal neovascularization by greater than 90% when compared to controls. Further, these effects were shown to be dose dependent such that, in vivo, inhibition of neovascular growth was induced at doses as low as 0.5 μg/Kg to 5 μg/Kg, doses which tended to stimulate EC cell proliferation in vivo. Thus, calcitriol in doses that have been found to be therapeutically effective can be used to inhibit neovascular growth, particularly in the retina. Therefore, systemic administration of calcitriol may be used as an efficacious treatment for non-neoplastic neovascular growth such as that exhibited in diabetic retinopathy, retinopathy of hypertension and wet macular degeneration.

Without being held to any particular theory, this data strongly suggests that calcitriol may exert its effects on cell growth and differentiation by, at least, two different mechanisms: one mechanism which results in an increase in cell proliferation at low doses and further has no effect on proliferation at high doses; and another mechanism which, while having no effect on proliferation, has a profound effect on capillary morphogenesis. While such mechanisms are not fully understood, these data may illustrate the effects of calcitriol that are independent of the vitamin D receptor or effects that are masked by calcium toxicity resulting from the high serum calcium concentration due to dosing animals with excessive amounts of calcitriol.

Further, it should be noted that, while in the studies described herein calcitriol was administered systemically by intraperitoneal injection, the route of calcitriol administration can be made by any effective means, as discussed previously. For example, it may be appreciated that, in some instances, the vitamin D compound of the invention is administered directly into the eye by means of drops, ophthalmic cream, a hydrogel or the like placed in the eye or under the eyelid. In addition, where the neovascular growth is superficial, such as, for example, a vascular birthmark, the vitamin D compound of the invention is administered topically as a cream or salve.

While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

What is claimed is:

1. A method for inhibiting retinal endothelial cell capillary morphogenesis, or inhibiting retinal angiogenesis in a subject in need thereof, the method comprising administering to the subject a suitable pharmaceutical composition comprising an amount of vitamin D effective for inhibiting retinal angiogenesis to a subject in need thereof.

2. A method for treating a disease whose pathological manifestation is dependent on angiogenesis in a subject in need thereof, comprising administering to the subject a suitable pharmaceutical composition comprising an amount of vitamin D effective for inhibiting angiogenesis to a subject in need thereof.

3. A method of treating in a subject in need thereof a pathological condition resulting from angiogenesis of the eye, or treating a primitive neuroectodermal tumor, the method comprising administering to the subject a suitable pharmaceutical composition comprising an amount of vitamin D effective for inhibiting angiogenesis.

4. The method of claim 3, wherein the pathological condition is a non-neoplastic eye disease, or a neoplastic eye disease excluding retinoblastoma.

5. The method according to claim 4, wherein the non-neoplastic eye disease has a choroidal neovascularization component, or retinal neovascularization component, or both.

6. The method according to claim 5, wherein the non-neoplastic eye disease is selected from the group consisting of ROP, AMD, diabetic retinopathy, hypertensive retinopathy, central retinal vein occlusion (CRVO), branch vein occlusion (BRVO), neovascular glaucoma, ocular ischemic syndrome, occlusive vasculitis, polypoidal choroidal vasculopathy, myopic choroidal neovascularization, radiation retinopathy, chorioretinitis, central serous choroidopathy, central retinal artery occlusion, uveitic macular edema, idiopathic juxtafoveal telangiectasia, angioid streaks, sickle cell retinopathy, and pseudophakic cystoid macular edema.

7. The method according to claim 5, wherein the neoplastic eye disease has retinal neovascularization as a key component.

8. The method according to claim 7, wherein the neoplastic eye disease is selected from the group consisting of a primary ocular tumor, retinal angioma, retinal glioma and astocytoma, choroidal hemangioma, choroidal neurofibroma, choroidal hamartoma, choristomas, ocular lymphoma, ocular phakomatosis, and metastatic ocular tumors related to choroidal and retinal neovascularization.

9. The method according to claim 8, wherein primary ocular tumor is selected from the group consisting of uveal melanoma, melanocytoma, retinocytoma, retinal hamartoma and choristoma. Wherein the PNET affects the brain and spinal cord.

10. The method according to claim 3, wherein the primitive neuroectodermal tumors is medulloblastoma, pineoblastoma, non-pineal supratententorial, or Ewings sarcoma.

11. The method according to claim 3, wherein the vitamin D is ergocalciferol or cholecalciferol or a derivative thereof.

12. The method according to claim 3, wherein the vitamin D is selected from the group consisting of calcitriol, 1α,24-dihydroxy vitamin D, α-calcidol, calcifedol, 1α,24,25-trihydroxy vitamin D, 1β,25-dihydroxy vitamin D, 22-oxacalcitriol, calcipotriol, and dihydrotachysterol.

13. The method according to claim 12, wherein the vitamin D is calcitriol, or a salt or prodrug thereof.

14. The method of claim 3, wherein the calcitriol is administered systemically.

15. The method of claim 3, wherein the subject is a human.
